(19) United States
(12) Patent Application Publication
(10) Pub. No.: US 2023/0024991 A1
(43) Pub. Date: Jan. 26, 2023

(54) PRODRUGS OF ACYL CARNITINES

(71) Applicant: ATAI Life Sciences AG, Berlin (DE)

(72) Inventors: Robert B. PERNI, Marlborough, MA (US); Glenn SHORT, Scituate, MA (US); Srinivas RAO, Encinitas, CA (US)

(21) Appl. No.: 17/848,015

(22) Filed: Jun. 23, 2022

Related U.S. Application Data
(60) Provisional application No. 63/214,065, filed on Jun. 23, 2021.

(51) Int. Cl. C07C 229/22

(52) U.S. Cl. CPC C07C 229/22

(57) ABSTRACT

Provided herein are prodrugs comprising acyl carnitines including those of Formula (I):

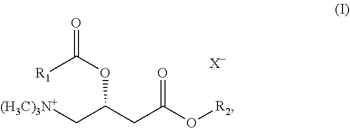

wherein:
R₁ is alkyl or alkenyl;
R₂ is alkyl or aryl; and
X⁻ is a pharmaceutically acceptable anion.

### CROSS-REFERENCE TO RELATED APPLICATIONS

The present application claims priority to U.S. Provisional Application Ser. No. 63/214,065, filed on Jun. 23, 2021, and which is hereby incorporated by reference in its entirety.

### BACKGROUND

Carnitine is a water-soluble small molecule featuring in a number of essential roles in intermediary metabolism. The primary physiological role is associated with cellular energy-producing processes through the transport of long-chain fatty acids from the cytosol into the mitochondria, where their degradation takes place via β-oxidation. This role is fundamental, since neither free long chain fatty acids, nor their coenzyme-A (CoA) esters can cross the inner mitochondrial membrane on their own i.e., the transport is possible exclusively in a carnitine-ester form. The main pathway for the degradation of fatty acids in the mitochondria is β-oxidation, which produces acetyl-CoA as the end-product, a key metabolic pathway for energy homoeostasis in tissues such as liver, heart and skeletal muscle. Finally, either the acetyl-CoA is used directly for the generation of energy (through Krebs cycle) or they are converted to acetylcarnitine (ACL) for transport out of the mitochondria to be used elsewhere, for example, in synthesizing lipids, maintaining membrane composition, increasing antioxidant activity, and enhancing cholinergic neurotransmission. Acylcarnitine deficiencies have been implicated, for example, in neuropsychiatric diseases such as depression. Administration of acylcarnitines could provide effective new approaches for the treatment of depression and other neuropsychiatric diseases where mitochondrial energy defects are linked to disease pathology.

There remains a need for improved acylcarnitine prodrugs.

### SUMMARY

In one aspect, the present disclosure provides a compound of Formula (I):

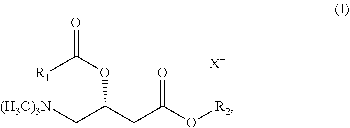

wherein:

R₁ is alkyl, alkenyl, alkynyl or aryl;

R₂ is alkyl, alkenyl, alkynyl or aryl; and

X⁻ is a pharmaceutically acceptable anion.

In some embodiments, the present disclosure provides a compound of Formula (II):

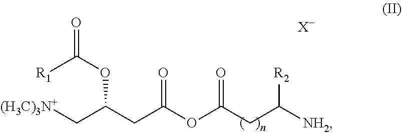

wherein:

R₁ is alkyl, alkenyl, alkynyl or aryl;

R₂ is H, COOH, COORₐ, or CONR_{b}R_{c};

Rₐ is alkyl, cycloalkyl, aryl or heteroaryl;

R_{b} and R_{c} are independently hydrogen, alkyl, cycloalkyl, aryl or heteroaryl;

n is an integer from 2-5; and

X⁻ is a pharmaceutically acceptable anion.

In some embodiments, the present disclosure provides a compound of Formula (III):

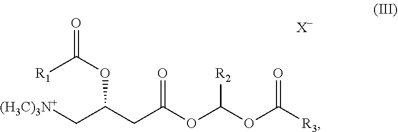

wherein:

R₁ is alkyl, alkenyl, alkynyl or aryl;

R₂ is alkyl, cycloalkyl, aryl or heteroaryl;

R₃ is alkyl, cycloalkyl, aryl or heteroaryl; and

X⁻ is a pharmaceutically acceptable anion.

In some embodiments, the present disclosure provides a compound of Formula (IV):

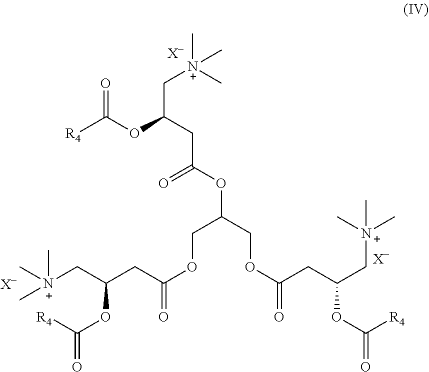

wherein, each R₄ is independently C₁₋₆ alkyl, alkynyl or aryl, and

X⁻ is a pharmaceutically acceptable anion.

In some embodiments, the present disclosure provides a pharmaceutical composition comprising a compound of Formula (I), (II), (III), (IV), or a pharmaceutically acceptable salt thereof and a pharmaceutically acceptable excipient.

In some embodiments, the present disclosure provides a method of treating a mental health disease or disorder, the method comprising administering to a subject in need thereof a therapeutically effective amount of one of the compounds (I), (II), (III) or (IV) or a pharmaceutically acceptable salt thereof, or a composition including those compounds.

In some embodiments, the present disclosure provides a method of treating a mental health disease or disorder including a major depressive disorder, treatment resistant depression, substance use disorders or eating disorders, the method comprising administering to a subject in need thereof a therapeutically effective amount of one of the compounds (I), (II), (III) or (IV) or a pharmaceutically acceptable salt thereof, or a composition including those compounds.

In some embodiments, the present disclosure provides a method of treatment a mental health disease or disorder including depression, including treatment resistant depression and bipolar depression; dysthymia; fibromyalgia; chronic fatigue; anxiety; sexual dysfunction; multiple sclerosis; anhedonia associated with substance use disorders including alcohol use disorder, dependence and/or withdrawal, pain, including acute and chronic conditions, including neuropathies; cardiac disease; primary carnitine deficiency; secondary carnitine deficiency, including renal disease or drug treatment; inborn errors/metabolic disease; intermittent claudication; or peripheral artery disease, the method comprising administering to a subject in need thereof a therapeutically effective amount of one of the compounds (I), (II), (III) or (IV) or a pharmaceutically acceptable salt thereof, or a composition including those compounds.

In some embodiments, the present disclosure provides a prodrug composition including one of the compounds of the formula (I), (II), (III) or (IV) which breaks down into a carnitine in an amount effective to treat major depressive disorder or treatment resistant disorder, such as 50-500 mg/day.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to forty carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and Cm alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkenyl” or “alkenyl group” refers to a straight or branched hydrocarbon chain having from two to forty carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain having from two to forty carbon atoms, and having one or more carbon-carbon triple bonds. Each alkynyl group is attached to the rest of the molecule by a single bond. Alkynyl group comprising, for example, any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiphenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the groups described herein (e.g., alkyl, alkenyl, alkynyl, alkoxy, aryl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

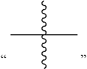

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

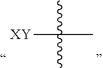

indicates that the chemical entity “XY” is bonded to another chemical entity via the point of attachment bond. Furthermore, the specific point of attachment to the nondepicted-chemical entity can be specified by inference. For example, the compound CH₃—R^{3X}, wherein R^{3X} is H or

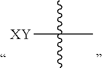

infers that when R^{3X} is “XY”, the point of attachment bond is the same bond as the bond by which R^{3X} is depicted as being bonded to CH₃.

### Compounds

The present disclosure provides compounds that are acylcarnitine prodrugs as well as pharmaceutical compositions thereof.

Mitochondria are organelles within the cell that generate the energy that sustains adenosine triphosphate (ATP) production, which is necessary for cell activity and survival. The oxidation of metabolites occurs in the mitochondria, through the Krebs' cycle and through the β-oxidation of fatty acids. Mitochondria are also the main generators of reactive oxygen species (ROS) and calcium homeostasis. They regulate cellular pathways, including the release of neurotransmitters from neurons and glial cells. Mitochondria also respond to perturbations in cell homeostasis during stress. Mitochondrial dysfunction has been implicated in human neurodegenerative and neuropsychiatric diseases. A major role for energy production in the mitochondria is a result of β-oxidation of fatty acids. This process is facilitated by carnitine and its acyl derivatives.

Carnitine is a water-soluble small molecule featuring in a number of essential roles in intermediary metabolism. The primary physiological role is associated with cellular energy-producing processes through the transport of long-chain fatty acids from the cytosol into the mitochondria, where their degradation takes place via β-oxidation. This role is fundamental, since neither free long chain fatty acids, nor their coenzyme-A (CoA) esters can cross the inner mitochondrial membrane on their own i.e., the transport is possible exclusively in a carnitine-ester form. The main pathway for the degradation of fatty acids in the mitochondria is β-oxidation, which produces acetyl-CoA as the end-product, a key metabolic pathway for energy homoeostasis in tissues such as liver, heart and skeletal muscle. Finally, either the acetyl-CoA is used directly for the generation of energy (through Krebs cycle) or they are converted to acetylcarnitine (ACL) for transport out of the mitochondria to be used elsewhere, for example, in synthesizing lipids, maintaining membrane composition, increasing antioxidant activity, and enhancing cholinergic neurotransmission. The metabolic profiles of ACLs provide readouts of mitochondrial function, energetics and metabolic states. As the acylation state of carnitine in the plasma reflects the composition of the cytosolic acylcarnitine pool, this serves as a diagnostic marker for the equilibrium between acyl-CoA and acylcarnitine species. As a consequence, abnormal concentrations ACL or altered distribution of ACL chain lengths may be indicative of particular disorders or provide for a precision medicine approach to gauge responsivity to certain therapeutic interventions a priori.

A study reported by Nasca et al in 2018 found that C2 acylcarnitine (where C2 denotes a carbon chain length of two), was lower in patients with major depressive disorder (MDD, n=71) compared to healthy controls (n=45) (Nasca, C. et al. Acetyl-l-carnitine deficiency in patients with major depressive disorder. Proc National Acad Sci 115, 201801609 (2018)). Another study in 2015 noted decreased ACLs (C3, C4, C5 and C12) in plasma and distinguished depressed subjects from controls in HIV-positive and HIV-negative cohorts, and these alterations correlated with the severity of depressive symptoms (Cassol, E. et al. Altered Monoamine and Acylcarnitine Metabolites in HIV-Positive and HIV-Negative Subjects With Depression. Jaids J Acquir Immune Defic Syndromes 69, 18-28 (2015). Several studies have also reported perturbations in C2 levels after antidepressant treatment in depressed patients (Nasca, C. et al. Acetyl-l-carnitine deficiency in patients with major depressive disorder. Proc National Acad Sci 115, 201801609 (2018); Moaddel, R. et al. Plasma metabolomic profiling of a ketamine and placebo crossover trial of major depressive disorder and healthy control subjects. Psychopharmacology 235, 3017-3030 (2018); and Rotroff, D. M. et al. Metabolomic signatures of drug response phenotypes for ketamine and esketamine in subjects with refractory major depressive disorder: new mechanistic insights for rapid acting antidepressants. Transl Psychiat 6, e894-e894 (2016). Further, in a rat model of depression, incomplete β-oxidation of fatty acids has been associated with elevated medium- and long-chain ACLs. A more recent study in participants with MDD (n=136) reported decreased C2, medium- and long-chain ACLs after 8 weeks of antidepressant (citalopram/escitalopram) therapy (Mahmoudian Dehkordi, S. et al. Alterations in Acylcarnitines, Amines, and Lipids Inform about Mechanism of Action of Citalopram/Escitalopram in Major Depression. Biorxiv 2020.02.10.927012 (2020) doi:10.1101/2020.02.10.927012) suggesting that the drug may act to restore the mitochondrial β-oxidation processes with greater utilization of the medium and long-chain acylcarnitines (Chen, S. et al. Effect of Allium macrostemon on a rat model of depression studied by using plasma lipid and acylcarnitine profiles from liquid chromatography/mass spectrometry. J Pharmaceut Biomed 89, 122-129 (2014). The same study also reported significant increase in other short chain acylcarnitines (C3, C4 and C5).

Studies have also suggested a role for ACLs in depression and response to treatment. For example, baseline levels of short chain acylcarnitine have been shown to inform about treatment outcomes (Ahmed, A. T. et al. Acylcarnitine Metabolomic Profiles Inform Clinically-Defined Major Depressive Phenotypes. J Affect Disorders 264, 90-97 (2019)). In addition, metabolomics and linked genetic analysis highlighted key enzymes that seem implicated in subtypes of depression and resistance to treatment. Drugs that target this system when used in combination with other drugs that target mitochondrial energetics could provide effective new approaches for treatment of depression that do not respond to currently used drugs. A novel precision medicine approach using -omics data will guide patient stratification and tailoring of specific acylcarnitine therapies that may help to normalize mitochondrial energetics and reduce depression symptoms. Other neuropsychiatric diseases will benefit from such approaches as they too have mitochondrial energy defects linked to disease pathology.

A recent systematic and meta-analytic review has suggested that ACL administration leads to a significant and clinically meaningful reduction in depression compared with placebo/no intervention (Veronese N, Stubbs B, Solmi M, Ajnakina O, Carvalho A F, Maggi S. Acetyl-L-Carnitine Supplementation and the Treatment of Depressive Symptoms: A Systematic Review and Meta-Analysis. Psychosom Med. 2018 February/March; 80(2):154-159. Acylcarnitine treatment has been suggested using supplementation with ACL (Nasca, C. et al. Acetyl-l-carnitine deficiency in patients with major depressive disorder. Proc National Acad Sci 115, 201801609 (2018); Bigio, B. et al. Epigenetics and energetics in ventral hippocampus mediate rapid antidepressant action: Implications for treatment resistance. Proc National Acad Sci 113, 7906-7911 (2016). Studies on animal and cellular models have also suggested that ACL exerts neuroplastic effect, membrane modulation, and neurotransmitter regulation, and has a potential role as an antidepressant. Results from a placebo-controlled study in 1990 that evaluated the effects of ACL on senile patients suffering from depression (n=28), indicated that ACL is effective in counteracting symptoms of depression in the elderly (Garzya, G. et al. Evaluation of the effects of L-acetylcarnitine on senile patients suffering from depression. Drug Exp Clin Res 16, 101-6 (1990)).

In some embodiments, administration of acylcarnitine prodrugs of the present disclosure can restore acylcarnitine concentrations to levels typically associated with healthy individuals to therapeutically address any deficits in acylcarnatines, for example deficits in acylcarnatines underlying a neuropsychiatric disease, such as depression.

Intake and clearance of L-carnitine and acylcarnitines are tightly regulated to maintain baseline endogenous concentrations. The absorption of L-carnitine in the diet occurs mainly via carrier-mediated transport but also through passive absorption in the intestine. Carrier-mediated transport is fairly efficient, and the bioavailability of dietary L-carnitine depends on the amount in the meal but ranges from 54-87% (Rebouche, C J, and Chenard, C A Metabolic Fate of Dietary Carnitine in Human Adults: Identification and Quantification of Urinary and Fecal Metabolites, J. Nutr 1991 121: 539-54). L-carnitine is significantly absorbed only in the small intestine, without undergoing first-pass degradation, and in a dose-dependent manner (Matsuda, K., Yuasa, H., and Watanabe, J. Fractional absorption of L-carnitine after oral administration in rats: evaluation of absorption site and dose dependency, Biol Pharm Bull 1998 21: 752-755). Intestinal active transport is easily saturated and following oral administration of large amounts of exogenous carnitine (1-6 g), passive absorption is much less efficient and bioavailability drops to 5-18% (Evans, A. M., Fornasini, G. Pharmacokinetics of L-Carnitine, Clin Pharmacokinet 2003 42: 941-967). Unabsorbed L-carnitine is almost completely degraded by microorganisms in the large intestine. Absorption processes are similar for orally administered acylcarnitines; however, acylcarnitines are partially hydrolyzed by enterocytes upon absorption. Once in circulation, acylcarnitines are further hydrolyzed to yield L-carnitine (Marzo, A., Arrigoni Martelli, E., Urso, R. et al. Metabolism and disposition of intravenously administered acetyl-L-carnitine in healthy volunteers. Eur J Clin Pharmacol 1989 37: 59-63). Following orally administered of acetyl-L-carnitine at 2 g/day, circulating concentrations of acetyl-L-carnitine were increased 43%, indicating that not all of it is hydrolyzed upon absorption (Rebouche, C J, Kinetics, pharmacokinetics, and regulation of L-carnitine and acetyl-L-carnitine metabolism, Acad Sci. 2004; 1033:30-41). These carnitine compounds are cleared from the body through renal excretion. Under normal baseline conditions, L-carnitine undergoes extensively tubular reabsorption in the kidney reclaiming about 99%. This reabsorption in the kidney displays saturation kinetics; therefore, as circulating L-carnitine concentration increases, efficiency of reabsorption decreases and overall renal clearance increases (Evans, A. M., Fornasini, G. Pharmacokinetics of L-Carnitine, Clin Pharmacokinet 2003 42: 941-967). This results in rapid decline of circulating CAR concentration to baseline. In studies where exogenous acylcarnitines have been delivered IV, circulating concentrations of acylcarnitines and L-carnitine returned to baseline endogenous levels within 12 hours (Marzo, A., Arrigoni Martelli, E., Urso, R. et al. Metabolism and disposition of intravenously administered acetyl-L-carnitine in healthy volunteers. Eur J Clin Pharmacol 1989 37: 59-63).

In some embodiments, the acylcarnitine prodrugs of the present disclosure provide improved intestinal absorption but delay hydrolysis to carnitine. Such prodrugs can provide, for example, greater metabolic stability, and improvements in bioavailability and absorption, and provide greater plasma concentrations of acylcarnitines than would be available through simple dietary supplementation. Acylcarnitine prodrugs of the present disclosure may also decrease toxicity which may be associated with the parent drug or improve blood-brain barrier permeability delivery. Given that acylcarnitine must be delivered intracellularly, amino acid prodrugs of acylcarnitines of the present disclosure can improve active intracellular uptake via L-type amino acid transporters (LAT1 and LAT2).

In some embodiments, the present disclosure provides a compound of Formula (I):

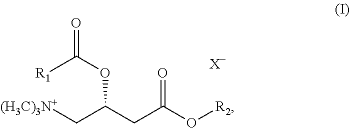

wherein:

R₁ is alkyl, alkenyl, alkynyl or aryl;

R₂ is alkyl, alkenyl, alkynyl or aryl; and

X⁻ is a pharmaceutically acceptable anion.

In some embodiments of the compounds of Formula (I), R₁ is alkyl.

In some embodiments of the compounds of Formula (I), R₁ is alkenyl.

In some embodiments of the compounds of Formula (I), R₁ is alkynyl.

In some embodiments of the compounds of Formula (I), R₁ is aryl.

In some embodiments of the compounds of Formula (I), R₁ is C₁-C₁₀ alkyl.

In some embodiments of the compounds of Formula (I), R₁ is C₁-C₁₀ alkenyl.

In some embodiments of the compounds of Formula (I), R₁ is C₅-C₁₁ alkyl.

In some embodiments of the compounds of Formula (I), R₁ is CH(CH₂CH₂CH₃)₂.

In some embodiments of the compounds of Formula (I), R₁ is C₅-C₁₁ alkenyl.

In some embodiments of the compounds of Formula (I), R₁ is C₁₂-C₂₀ alkyl.

In some embodiments of the compounds of Formula (I), R₁ is C₁₂-C₂₀ alkenyl.

In some embodiments of the compounds of Formula (I), R₁ is a very long chain fatty acid alkyl (i.e., ≥22 carbons). In some embodiments, R₁ is C₂₂-C₃₉ alkyl. In some embodiments, R₁ is C₂₂-C₂₈ alkyl.

In some embodiments of the compounds of Formula (I), R₁ is a very long chain fatty acid alkenyl (i.e., ≥22 carbons). In some embodiments, R₁ is C₂₂-C₃₉ alkenyl. In some embodiments, R₁ is C₂₂-C₂₈ alkenyl.

In some embodiments of the compounds of Formula (I), R₂ is alkyl.

In some embodiments of the compounds of Formula (I), R₂ is aryl.

In some embodiments of the compounds of Formula (I), R₂ is methyl.

In some embodiments of the compounds of Formula (I), R₂ is ethyl.

In some embodiments of the compounds of Formula (I), R₂ is n-propyl.

In some embodiments of the compounds of Formula (I), R₂ is isopropyl.

In some embodiments, the present disclosure provides pharmaceutical compositions comprising a therapeutically effective amount of a compound of Formula (I) and a pharmaceutically acceptable carrier.

In some embodiments, the present disclosure provides a compound of Formula (II):

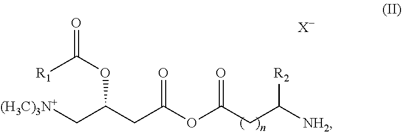

wherein:

R₁ is alkyl, alkenyl, alkynyl or aryl;

R₂ is H, COOH, COORₐ, or CONR_{b}R_{c};

Rₐ is alkyl, cycloalkyl, aryl or heteroaryl;

R_{b} and R_{c} are independently hydrogen, alkyl, cycloalkyl, aryl or heteroaryl;

n is an integer from 2-5; and

X⁻ is a pharmaceutically acceptable anion.

In some embodiments of the compounds of Formula (II), X is chloride, acetate, sulfate, phosphate, or maleate.

In some embodiments of the compounds of Formula (II), X is chloride.

In some embodiments of the compounds of Formula (II), R₁ is alkyl.

In some embodiments of the compounds of Formula (II), R₁ is alkenyl.

In some embodiments of the compounds of Formula (II), R₁ is alkynyl.

In some embodiments of the compounds of Formula (II), R₁ is aryl.

In some embodiments of the compounds of Formula (II), R₁ is C₁-C₄ alkyl.

In some embodiments of the compounds of Formula (II), R₁ is C₁-C₄ alkenyl.

In some embodiments of the compounds of Formula (II), R₁ is C₅-C₁₁ alkyl.

In some embodiments of the compounds of Formula (II), R₁ is C₅-C₁₁ alkenyl.

In some embodiments of the compounds of Formula (II), R₁ is CH(CH₂CH₂CH₃)₂.

In some embodiments of the compounds of Formula (II), R₁ is C₁₂-C₂₀ alkyl.

In some embodiments of the compounds of Formula (II), R₁ is C₁₂-C₂₀ alkenyl.

In some embodiments of the compounds of Formula (II), R₁ is a very long chain fatty acid alkyl (i.e., ≥22 carbons). In some embodiments, R₁ is C₂₂-C₃₆ alkyl. In some embodiments, R₁ is C₂₂-C₂₈ alkyl.

In some embodiments of the compounds of Formula (II), R₁ is a very long chain fatty acid alkenyl (i.e., ≥22 carbons). In some embodiments, R₁ is C₂₂-C₃₆ alkenyl. In some embodiments, R₁ is C₂₂-C₂₈ alkenyl.

In some embodiments of the compounds of Formula (II), R² is H, COOH, or COORₐ, or CONR_{b}R_{c}.

In some embodiments of the compounds of Formula (II), R₂ is hydrogen.

In some embodiments of the compounds of Formula (II), R₂ is COOH.

In some embodiments of the compounds of Formula (II), R² is CONR_{b}R_{c}.

In some embodiments of the compounds of Formula (II), Rₐ is alkyl, cycloalkyl, aryl or heteroaryl.

In some embodiments of the compounds of Formula (II), R_{b} and R_{c} are independently hydrogen, alkyl, cycloalkyl, aryl or heteroaryl.

In some embodiments of the compounds of Formula (II), n is an integer from 2-5 (i.e. 2, 3, 4, or 5). In some embodiments, n is 2. In some embodiments n is 3. In some embodiments, n is 4. In some embodiments, n is 4. In some embodiments, n is 5.

In some embodiments, the present disclosure provides pharmaceutical compositions comprising a therapeutically effective amount of a compound of Formula (II) and a pharmaceutically acceptable carrier

In some embodiments, the present disclosure provides a compound of Formula (III):

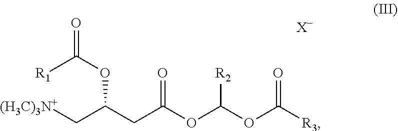

R₁ is alkyl, alkenyl, alkynyl or aryl;

R₂ is alkyl, cycloalkyl, aryl or heteroaryl;

R₃ is alkyl, cycloalkyl, aryl or heteroaryl; and

X⁻ is a pharmaceutically acceptable anion.

In some embodiments of the compounds of Formula (III), X is chloride, acetate, sulfate, phosphate, and maleate.

In some embodiments of the compounds of Formula (III), X is chloride.

In some embodiments of the compounds of Formula (III), R₁ is alkyl.

In some embodiments of the compounds of Formula (III), R₁ is alkenyl.

In some embodiments of the compounds of Formula (III), R₁ is alkynyl.

In some embodiments of the compounds of Formula (III), R₁ is aryl.

In some embodiments of the compounds of Formula (III), R₁ is C₁-C₄ alkyl.

In some embodiments of the compounds of Formula (III), R₁ is C₁-C₄ alkenyl.

In some embodiments of the compounds of Formula (III), R₁ is C₅-C₁₁ alkyl.

In some embodiments of the compounds of Formula (III), R₁ is C₅-C₁₁ alkenyl.

In some embodiments of the compounds of Formula (III), R₁ is CH(CH₂CH₂CH₃)₂.

In some embodiments of the compounds of Formula (III), R₁ is C₁₂-C₂₀ alkyl.

In some embodiments of the compounds of Formula (III), R₁ is C₁₂-C₂₀ alkenyl.

In some embodiments of the compounds of Formula (III), R₁ is a very long chain fatty acid alkyl (i.e., ≥22 carbons). In some embodiments, R₁ is C₂₂-C₃₆ alkyl. In some embodiments, R₁ is C₂₂-C₂₈ alkyl.

In some embodiments of the compounds of Formula (III), R₁ is a very long chain fatty acid alkenyl (i.e., ≥22 carbons). In some embodiments, R₁ is C₂₂-C₃₆ alkenyl. In some embodiments, R₁ is C₂₂-C₂₈ alkenyl.

In some embodiments of the compounds of Formula (III), R₂ is alkyl.

In some embodiments of the compounds of Formula (III), R₂ is methyl.

In some embodiments of the compounds of Formula (III), R₂ is isopropyl.

In some embodiments of the compounds of Formula (III), R₂ is tert-butyl.

In some embodiments of the compounds of Formula (III), R₂ is carbocycle.

In some embodiments of the compounds of Formula (III), R₂ is cyclopentyl.

In some embodiments of the compounds of Formula (III), R₂ is aryl.

In some embodiments of the compounds of Formula (III), R₂ is phenyl.

In some embodiments of the compounds of Formula (III), R₃ is alkyl.

In some embodiments of the compounds of Formula (III), R₃ is aryl.

In some embodiments, the present disclosure provides pharmaceutical compositions comprising a therapeutically effective amount of a compound of Formula (III) and a pharmaceutically acceptable carrier.

In some embodiments the present disclosure provides a compound of Formula (IV):

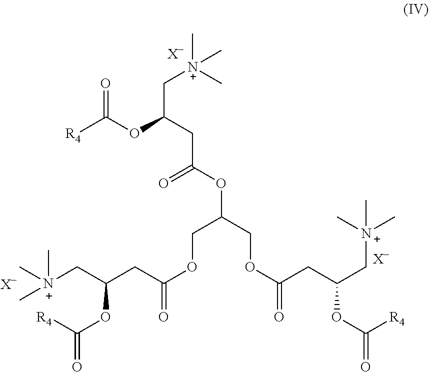

wherein, each R₄ is independently C₁₋₆ alkyl, and

X⁻ is a pharmaceutically acceptable anion.

In some embodiments of the compounds of Formula (IV), R₄ is C₁₋₄ alkyl,

In some embodiments the compound of Formula (IV) is:

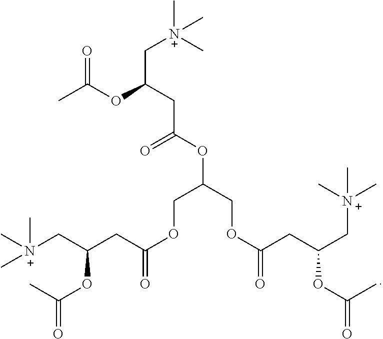 [SMILES: CC(=O)O[C@H](CC(=O)OCC(COC(=O)C[C@H](C[N+](C)(C)C)OC(C)=O)OC(=O)C[C@H](C[N+](C)(C)C)OC(C)=O)C[N+](C)(C)C]

In some embodiments, the compound of Formula (IV) is:

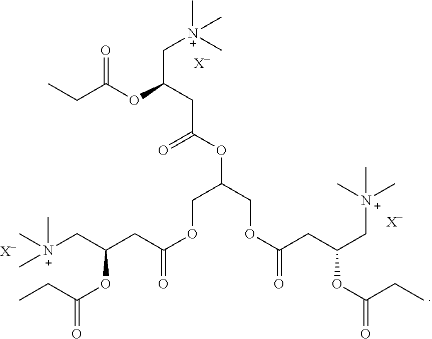

In some embodiments, the compound of Formula (IV) is:

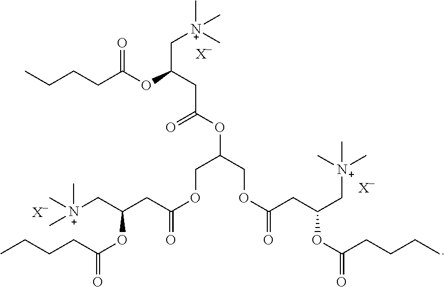

In some embodiments, the present disclosure provides pharmaceutical compositions comprising a therapeutically effective amount of a compound of Formula (IV) and a pharmaceutically acceptable carrier.

In some embodiments, provided herein is one or more compounds selected from Table 1.

In some embodiments, provided herein is one or more pharmaceutically acceptable salts of a compound selected from Table 1.

In some embodiments, provided herein is one or more compounds selected from Table 2.

In some embodiments, provided herein is one or more pharmaceutically acceptable salts of a compound selected from Table 2.

In some embodiments, provided herein is one or more pharmaceutically acceptable salts of a compound selected from Table 3.

In some embodiments, provided herein is a method of treating a mental health disease or disorder comprising administering a therapeutically effective amount of one of the above compounds.

In some embodiments, the mental health disease or disorder is a major depressive disorder, treatment resistant depression, substance use disorders or eating disorders.

In some embodiments, the mental health disease or disorder is depression, including treatment resistant depression and bipolar depression; dysthymia; fibromyalgia; chronic fatigue; anxiety; sexual dysfunction; multiple sclerosis; anhedonia associated with substance use disorders including alcohol use disorder, dependence and/or withdrawal, pain, including acute and chronic conditions, including neuropathies; cardiac disease; primary carnitine deficiency; secondary carnitine deficiency, including renal disease or drug treatment; inborn errors/metabolic disease; intermittent claudication; or peripheral artery disease.

TABLE 1

| Compound | | | |
|---|---|---|---|
| No. | Structure | Counteranion | Human Mean t_{1/2} (min) |
| 1 | 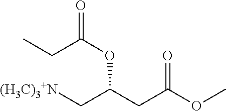 | Cl⁻ | 285.1 |
| 2 | 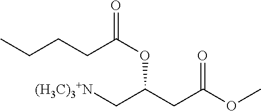 | Cl⁻ | 41.2 |
| 3 | 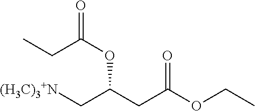 | Cl⁻ | 549.6 |
| 4 | 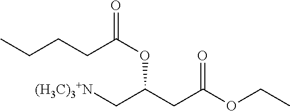 | Cl⁻ | 55.6 |
| 5 | 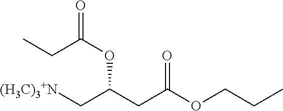 | Cl⁻ | 253.2 |
| 6 | 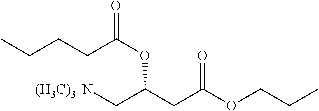 | Cl⁻ | 26.1 |
| 7 | 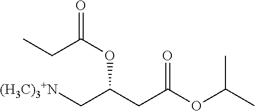 | I⁻ | 243.3 |
| 8 | 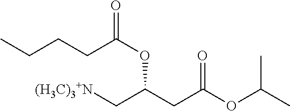 | Cl⁻ | 25.0 |

TABLE 2

| Com- | |
|---|---|
| pound | |
| No. | Structure |
| 9 | 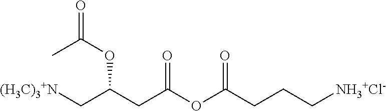 |
| 10 | 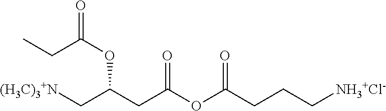 |
| 11 | 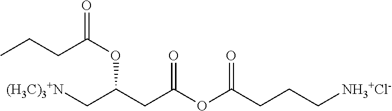 |
| 12 | 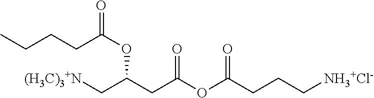 |

TABLE 3

| Compound | |
|---|---|
| No. | Structure |
| 13 | 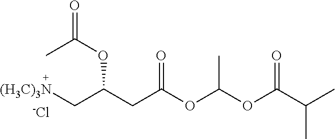 |
| 14 | 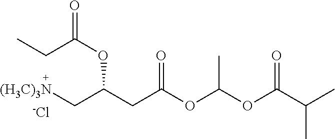 |
| 15 | 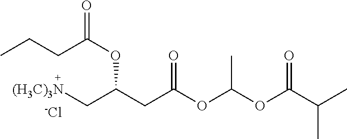 |
| 16 | 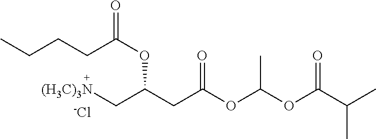 |

### Compositions

In some embodiments of the present disclosure, a pharmaceutical composition comprises a therapeutically effective amounts of one or more compounds of the present disclosure (e.g., a compound of Formula (I), (II), (III), (IV), Table 1, Table 2 or Table 3 or a pharmaceutically acceptable salt thereof) and a pharmaceutically acceptable excipient. In some embodiments, the present disclosure provides a pharmaceutical composition comprising a therapeutically effective amount of one or more compounds of the present disclosure (e.g., a compound of Formula (I), (II), (III), (IV), Table 1 or Table 2) and a pharmaceutically acceptable excipient.

The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

### EXAMPLES

The following examples, which are included herein for illustration purposes only, are not intended to be limiting.

Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. ¹HNMR (400 MHz) spectra was recorded on Broker spectrometers at RT with TMS or the residual solvent peak as the internal standard. Chemical shifts are given in (δ) and the coupling constants (J) are given as absolute values in Hertz (Hz). The multiplicities in ¹HNMR spectra are abbreviated as follows: s (singlet), d (doublet), t (triplet), q (quartet), m (multiplet), br or broad (broadened).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

Compounds of the present disclosure can be synthesized, for example, according to Schemes 1˜4 shown below.

Schemes 1-4: Representative Synthesis of Compounds of the Disclosure in Table 1.

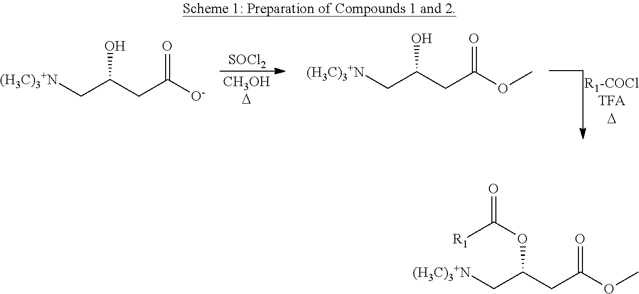

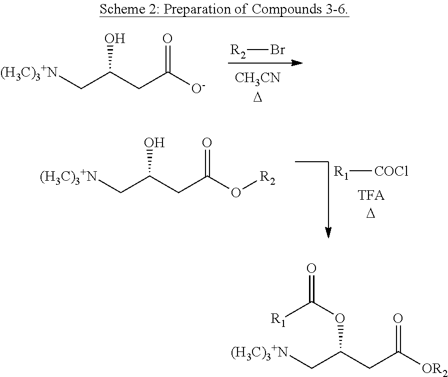

### Example 1: Preparation of (R)-4-ethoxy-N,N,N-trimethyl-4-oxo-2-(propionyloxy)butan-1-aminium chloride (3)

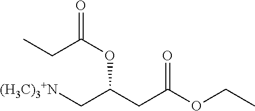

### Step 1: (R)-4-ethoxy-N,N,N-trimethyl-4-oxo-2-(propionyloxy)butan-1-aminium chloride (3)

To a solution of (R)-3-hydroxy-4-(trimethylammonio)butanoate (5 g, 31.02 mmol) in acetonitrile (50 mL) was added bromoethane (5.1 g, 46.53 mmol) and the resulting mixture was refluxed under hydrogen atmosphere for 60 hours. The reaction was monitored by proton NMR analysis until the starting material was consumed. The reaction mixture was concentrated to give a crude product which was triturated with diethyl ether (100 mL) and filtered. The filter cake was dried to afford (R)-4-ethoxy-2-hydroxy-N,N,N-trimethyl-4-oxobutan-1-aminium bromide (7 g, 84%) as a white solid. The product is very hydroscopic. ¹H NMR (400 MHz, D₂O) δ 4.70-4.62 (m, 1H), 4.19-4.13 (m, 2H), 3.46-3.44 (m, 2H), 3.19 (s, 9H), 2.69-2.56 (m, 2H), 1.23 (t, J=7.0 Hz, 3H).

### Step 2

To a solution of (R)-4-ethoxy-2-hydroxy-N,N,N-trimethyl-4-oxobutan-1-aminium bromide (2 g, 7.4 mmol) in trifluoroacetic acid (10 mL) was added propionyl chloride (2.1 g, 22 mmol) and the resulting mixture was stirred at 40° C. under hydrogen atmosphere for 60 hours. The reaction mixture was concentrated. The crude residue was triturated with diethyl ether (200 mL) for 2 hours and filtered. The filter cake was collected and dried to afford (R)-4-ethoxy-N,N,N-trimethyl-4-oxo-2-(propionyloxy)butan-1-aminium chloride (3) (1.9 g, 91%) as a white solid. The product is very hydroscopic. ¹H NMR (400 MHz, D₂O) δ 5.68-5.63 (m, 1H), 4.17-4.12 (m, 2H), 3.91-3.85 (m, 1H), 3.67-3.64 (m, 1H), 3.17 (s, 9H), 2.81 (d, J=6.0 Hz, 2H), 2.45-2.39 (m, 2H), 1.22 (t, J=7.2 Hz, 3H), 1.06 (t, J=7.4 Hz, 3H).

### Example 2. Preparation of (R)-4-ethoxy-N,N,N-trimethyl-4-oxo-2-(pentanoyloxy)butan-1-aminium chloride (4)

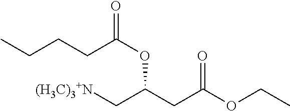

To a solution of (R)-4-ethoxy-2-hydroxy-N,N,N-trimethyl-4-oxobutan-1-aminium bromide as prepared in Step 1 above (2 g, 7.4 mmol) in trifluoroacetic acid (10 mL) was added pentanoyl chloride (2.7 g, 22 mmol) and the resulting mixture was stirred at 40° C. under hydrogen atmosphere for 60 hours. The reaction mixture was concentrated. The crude residue was added to diethyl ether (200 mL) slowly and stirred for 2 hours. The solid precipitate was collected by filtration and dried to afford (R)-4-ethoxy-N,N,N-trimethyl-4-oxo-2-(pentanoyloxy)butan-1-aminium chloride (4) (500 mg, 22%) as a white solid. The product is very hydroscopic. ¹H NMR (400 MHz, D₂O) δ 5.69-5.64 (m, 1H), 4.18-4.12 (m, 2H), 3.92-3.86 (m, 1H), 3.69-3.65 (m, 1H), 3.18 (s, 9H), 2.83-2.81 (m, 2H), 2.42 (t, J=7.4 Hz, 2H), 1.58-1.51 (m, 2H), 1.33-1.25 (m, 2H), 1.23 (t, J=7.0 Hz, 3H), 0.85 (t, J=7.4 Hz, 3H).

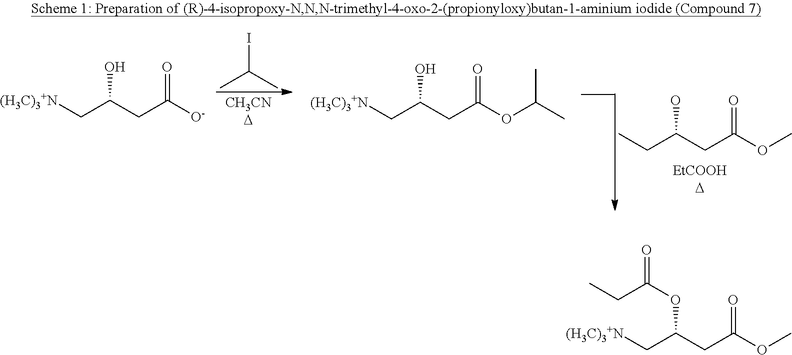

### Example 3

### Step 1

To a stirred solution of (R)-3-hydroxy-4-(trimethylammonio) butanoate (1 g, 6.2 mmol) in acetonitrile (10 mL) was added 2-iodopropane (4.14 g, 24.8 mmol) dropwise slowly. The resulting mixture was stirred at 90° C. overnight. After the reaction was completed, the mixture was cooled to room temperature and concentrated to give crude product (2.4 g, >100% yield) as a brown oil. ¹H NMR (400 MHz, CDCl₃) δ 4.97-5.06 (m, 1H), 4.74-4.79 (m, 1H), 3.95 (dd, J=1.2 Hz, J=6.6 Hz, 1H), 3.69 (dd, J=10 Hz, J=6.6 Hz, 1H), 3.52 (s, 9H), 2.61-3.76 (m, 2H), 1.26 (dd, J=2 Hz, J=3.2 Hz, 6H).

### Step 2

To a stirred solution of (R)-2-hydroxy-4-isopropoxy-N, N, N-trimethyl-4-oxobutan-1-aminium (1.05 g, 5.1 mmol) in propionic acid (3.8 g) was added propionic anhydride (2.67 g, 20.6 mmol). The resulting mixture was stirred at 80° C. overnight. After the reaction was completed, the mixture was cooled to room temperature and concentrated to give crude a crude residue which was taken up in acetone (50 mL) and stirred for 2 h at 0° C. The resulting precipitate was collected by filtration, rinsed with acetone and dried to afford the title compound (7) (210 mg, 11%) as a yellow solid. ¹H NMR (400 MHz, CDCl₃) δ 5.68 (dd, J=6.8 Hz, J=7.2 Hz, 1H), 4.95-5.04 (m, 1H), 4.27 (d, J=14 Hz, 1H), 4.11 (dd, J=8.8 Hz, J=7.2 Hz, 1H), 3.52 (s, 9H), 2.74-2.87 (m, 2H), 2.39-2.42 (m, 2H), 1.24 (dd, J=2 Hz, J=3.2 Hz, 6H), 1.15 (t, J=7.6 Hz, 3H).

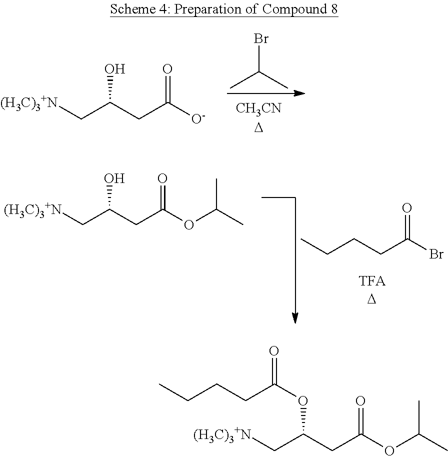

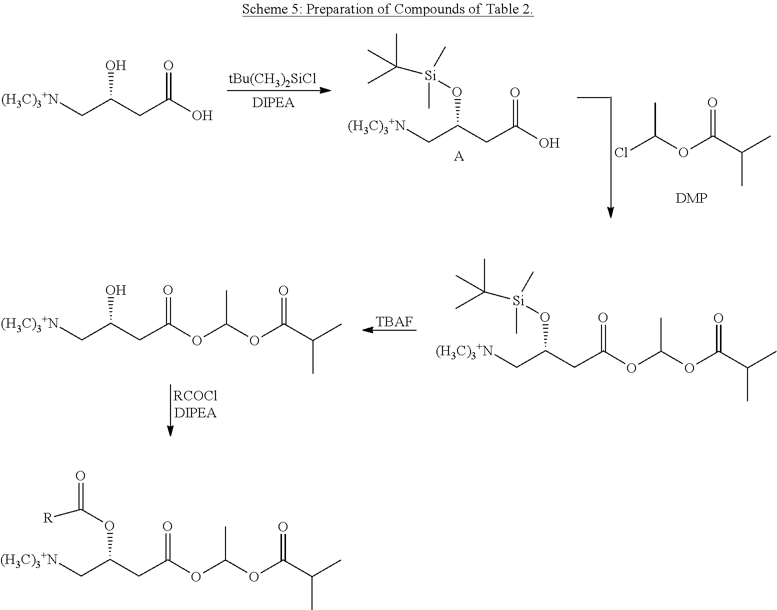

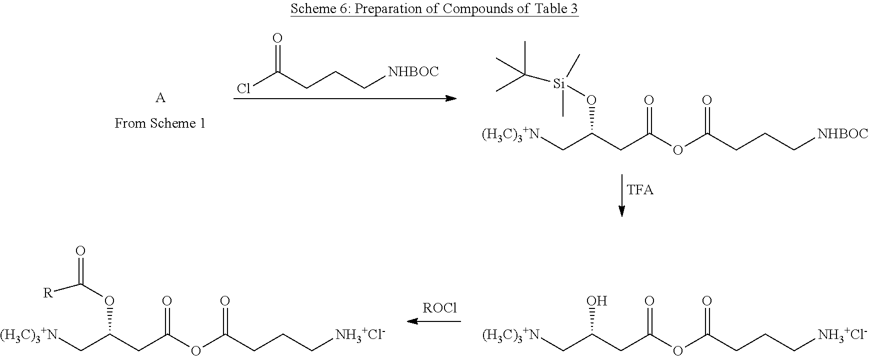

wherein R is alkyl, or alkenyl (e.g., methyl, ethyl, n-propyl, or n-butyl)

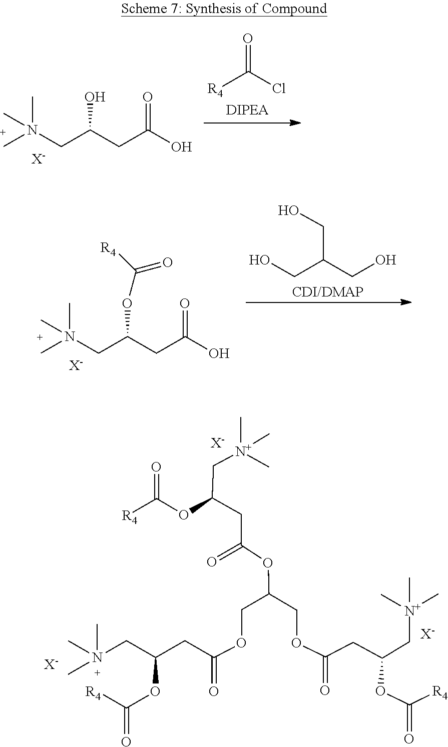

wherein R₄ is defined herein.

TABLE 4
DETERMINATION OF PLASMA HALF LIFE

| | Compound | Mean |
|---|---|---|
| Species | No. | t_{1/2} (min) |
| HUMAN | 1 | 285.1 |
| | 2 | 41.2 |
| | 3 | 549.6 |
| | 4 | 55.6 |
| | 5 | 253.2 |
| | 6 | 26.1 |
| | 7 | 243.3 |
| | 8 | 25.0 |
| RAT | 1 | 260.1 |
| | 2 | — |
| | 3 | 527.4 |
| | 4 | 259.6 |
| | 5 | 220.4 |
| | 6 | 66.3 |
| | 7 | 1114.9 |
| | 8 | 500.7 |
| | | t_{1/2} (min) |
| DOG | 1 | 195.4 |
| | 2 | 141.5 |
| | 3 | 1359.1 |
| | 4 | 701.8 |
| | 5 | 1219.0 |
| | 6 | 544.8 |
| | 7 | 1680.4 |
| | 8 | 414.7 |

Human, rat, and dog plasma were dialyzed against PBS buffer, pH 7.4 using Thermo Slide-A-Lyzer™ G2 Dialysis Cassettes for 24 hours at 4° C. prior to the experiment.

The assay was carried out in 96-well microtiter plates. Prodrugs and the reference compound (propantheline) at a final concentration of 1 μM were incubated separately in singlet at 37° C. with mouse, rat, dog, monkey or human plasma for 0, 30, 60, 120, 240, and 1440 min. At the end of each incubation time point, 300 μL of the quenching solutions (50% acetonitrile, 50% methanol, 0.05% formic acid) containing the internal standards (bucetin and d3-carnitine) was added to each well. The incubation plates were sealed, vortexed, and centrifuged at 10° C. for 15 minutes at 4000 rpm. The supernatant was transferred to fresh plates for LC/MS/MS analysis of the test compounds.

Sample Analysis

All samples were analyzed by LC/MS/MS using an AB Sciex API 4000 instrument (AB Sciex LLC, Framingham, Mass.), coupled to a Shimadzu LC-20AD LC Pump system (Shimadzu North America, Columbia, Md.). Some samples were separated using a Waters Atlantis T3 dC₁₈ reverse phase HPLC column (20 mm×2.1 mm) at a flow rate of 0.5 mL/min. The mobile phase consisted of 0.1% formic acid in water (solvent A) and 0.1% formic acid in 100% acetonitrile (solvent B). Other samples were separated using a Waters HILIC HPLC column (50 mm×2.1 mm) at a flow rate of 0.5 mL/min. The mobile phase consisted of 5 mM ammonium acetate in water (solvent A) and in 100% acetonitrile (solvent B).

MS Conditions

MS parameters for the test compounds are listed in Table 5.

TABLE 5

| | Q1 | Q3 | | | | |
|---|---|---|---|---|---|---|
| Compound | Mass | Mass | Polarity | DP* | CE* | CXP* |
| Acetylcarnitine | 204.2 | 85.1 | Positive | 25 | 25 | 10 |
| 1 | 232.2 | 99.1 | Positive | 55 | 25 | 10 |
| 2 | 260.3 | 99 | Positive | 50 | 25 | 10 |
| 3 | 246.2 | 85.2 | Positive | 50 | 35 | 10 |
| 4 | 274.2 | 85.2 | Positive | 60 | 35 | 10 |
| 5 | 260.3 | 85.1 | Positive | 60 | 45 | 10 |
| 6 | 288.2 | 85.1 | Positive | 70 | 35 | 10 |
| 7 | 260.3 | 85.2 | Positive | 55 | 35 | 10 |
| 8 | 288.3 | 85.2 | Positive | 45 | 35 | 10 |
| (R)-4-methoxy- | 176.2 | 117.1 | Positive | 25 | 25 | 10 |
| N,N,N- | | | | | | |
| trimethyl-4- | | | | | | |
| oxo-2- | | | | | | |
| (hydroxy)butan- | | | | | | |
| 1-aminium | | | | | | |
| chloride | | | | | | |
| (R)-4-ethoxy- | 190.2 | 103 | Positive | 50 | 25 | 10 |
| N,N,N- | | | | | | |
| trimethyl-4- | | | | | | |
| oxo-2- | | | | | | |
| (hydroxy)butan- | | | | | | |
| 1-aminium | | | | | | |
| chloride | | | | | | |
| (R)-4- | 204.2 | 103.1 | Positive | 55 | 25 | 10 |
| propyloxy- | | | | | | |
| N,N,N- | | | | | | |
| trimethyl-4- | | | | | | |
| oxo-2- | | | | | | |
| (hydroxy)butan- | | | | | | |
| 1-aminium | | | | | | |
| chloride | | | | | | |

*DP = declustering potential
CE = collision energy
CXP = collision cell exit potential

Data Analysis

The extent of metabolism was calculated based on the disappearance of prodrugs, compared to their initial concentration. The initial rates of clearance of prodrugs were calculated using linear regression plot of semi-log % remaining of prodrugs versus time. Assuming first order kinetics, the elimination rate constant, k, (equal to negative slope) of the linear regression plot was then used to determine t_{1/2} using the following formula: k=−slope; t_{1/2}=0.693/k.

### Numbered Embodiments of the Disclosure

In addition to the disclosure above, the Examples below, and the appended claims, the disclosure sets forth the following numbered embodiments.
1. A compound of Formula (I):

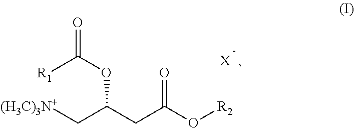

wherein:

R₁ is alkyl, alkenyl, alkynyl or aryl;

R₂ is alkyl, alkenyl, alkynyl, or aryl; and

X⁻ is a pharmaceutically acceptable anion.
2. The compound of embodiment 1, wherein R₁ is C₁-C₄ alkyl.
3. The compound of embodiment 1, wherein R₁ is C₅-C₁₁ alkyl.
4. The compound of embodiment 1, wherein R₁ is CH(CH₂CH₂CH₃)₂.
5. The compound of embodiment 1, wherein R₁ is C₁₂-C₂₀ alkyl.
6. The compound of embodiment 1, wherein R₁ is a long chain fatty acid alkyl including ≥21 carbons, or very long chain fatty acid alkenyl including ≥21 carbons.
7. The compound of embodiments 1 or 6, wherein R₁ is C₂₂-C₃₉ alkyl.
8. The compound of embodiment 1, wherein R₁ is alkenyl.
9. The compound of embodiment 1, wherein R₁ is alkynyl.
10. The compound of embodiment 1, wherein R₁ is aryl.
11. The compound of embodiments 1 or 10, wherein R₁ is phenyl.
12. The compound of any one of embodiments 1 to 11, wherein R₂ is methyl.
13. The compound of any one of embodiments 1 to 11, wherein R₂ is ethyl.
14. The compound of any one of embodiments 1 to 11, wherein R₂ is n-propyl.
15. The compound of any one of embodiments 1 to 11, wherein R₂ is isopropyl.
16. The compound of any one of embodiments 1 to 11, wherein R₂ is alkenyl.
17. The compound of any one of embodiments 1 to 11, wherein R₂ is alkynyl.
18. The compound of any one of embodiments 1 to 11, wherein R₂ is aryl.
19. The compound of any one of embodiments 1 to 11 or 18, wherein R₂ is phenyl.
20. The compound of embodiment 1, wherein the compound is selected from the group consisting of:

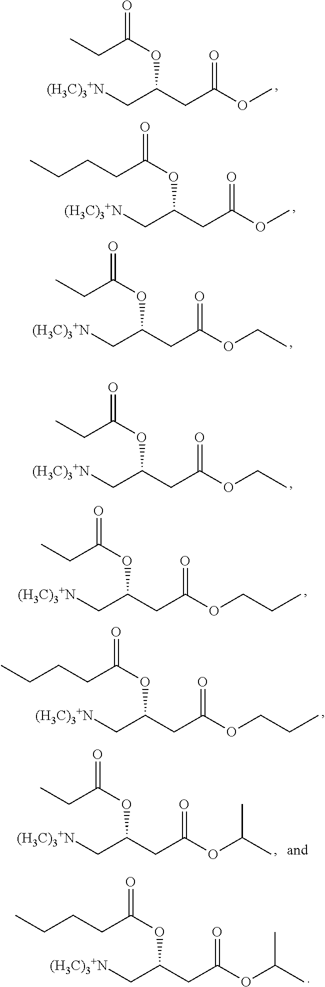

21. A compound of Formula (II):

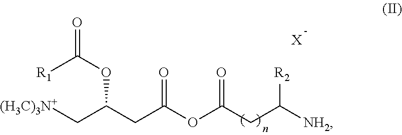

wherein:

R₁ is alkyl, alkenyl, alkynyl or aryl;

R₂ is H, COOH, COORₐ, or CONR_{b}R_{c};

Rₐ is alkyl, cycloalkyl, aryl or heteroaryl;

R_{b} and R_{c} are independently hydrogen, alkyl, cycloalkyl, aryl or heteroaryl;

n is an integer from 2-5; and

X⁻ is a pharmaceutically acceptable anion.
22. The compound of embodiment 21, wherein R₁ is C₁-C₄ alkyl.
23. The compound of embodiment 21, wherein R₁ is C₅-C₁₁ alkyl.
24. The compound of embodiment 21, wherein R₁ is CH(CH₂CH₂CH₃)₂.
25. The compound of embodiment 21, wherein R₁ is C₁₂-C₂₀ alkyl.
26. The compound of embodiment 21, wherein R₁ is a long chain fatty acid alkyl including ≥21 carbons.
27. The compound of embodiments 21 or 26, wherein R₁ is C₂₂-C₃₆ alkyl.
28. The compound of embodiment 21, wherein R₁ is alkenyl.
29. The compound of embodiment 21, wherein R₁ is alkynyl.
30. The compound of embodiment 21, wherein R₁ is aryl.
31. The compound of embodiment 21 or 30, wherein R₁ is phenyl.
32. The compound of any one of embodiments 21 to 31, wherein R₂ is hydrogen.
33. The compound of any one of embodiments 21 to 31, wherein R₂ is COOH.
34. The compound of any one of embodiments 21 to 31, wherein n is 2.
35. The compound of any one of embodiments 21 to 31, wherein n is 3
36. The compound of any one of embodiments 21 to 31, wherein n is 4.
37. The compound of any one of embodiments 21 to 31, wherein n is 5.
38. The compound of embodiment 21, wherein the compound is selected from the group consisting of:

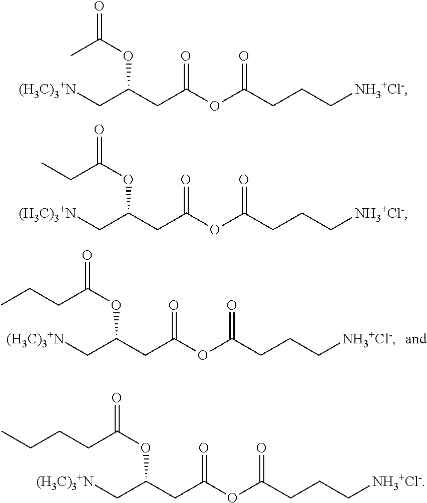

39. A compound of Formula (III):

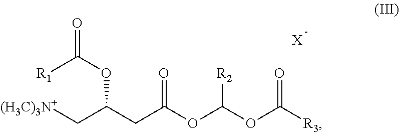

wherein:

R₁ is alkyl or aryl;

R₂ is alkyl, cycloalkyl, aryl or heteroaryl;

R₃ is alkyl, cycloalkyl, aryl or heteroaryl; and

X⁻ is a pharmaceutically acceptable anion.
40. The compound of embodiment 39, wherein R₁ is C₁-C₄ alkyl.
41. The compound of embodiment 39, wherein R₁ is C₅-C₁₁ alkyl.
42. The compound of embodiment 39, wherein R₁ is CH(CH₂CH₂CH₃)₂.
43. The compound of embodiment 39, wherein R₁ is C₁₂-C₂₀ alkyl.
44. The compound of embodiment 39, wherein R₁ is a fatty acid alkyl including ≥21 carbons.
45. The compound of embodiments 39 or 44, wherein R₁ is C₂₂-C₃₆ alkyl.
46. The compound of embodiment 39, wherein R₁ is aryl.
47. The compound of embodiments 39 or 46, wherein R₁ is phenyl.
48. The compound of any one of embodiments 39 to 47, wherein R₂ is alkyl.
49. The compound of any one of embodiments 39 to 47, wherein R₂ is methyl.
50. The compound of any one of embodiments 39 to 47, wherein R₂ is isopropyl.
51. The compound of any one of embodiments 39 to 47, wherein R₂ is tert-butyl.
52. The compound of any one of embodiments 39 to 47, wherein R₂ is cycloalkyl.
53. The compound of any one of embodiments 39 to 47, wherein R₂ is cyclopentyl.
54. The compound of any one of embodiments 39 to 47, wherein R₂ is aryl.
55. The compound of any one of embodiments 39 to 47 or 54, wherein R₂ is phenyl.
56. The compound of any one of embodiments 39 to 47, wherein R₂ is heteroaryl.
57. The compound of any one of embodiments 39 to 56, wherein R₃ is alkyl.
58. The compound of any one of embodiments 39 to 56, wherein R₃ is cycloalkyl.
59. The compound of any one of embodiments 39 to 56, wherein R₃ is aryl.
60. The compound of any one of embodiments 39 to 56 or 59, wherein R₃ is phenyl.
61. The compound of any one of embodiments 39 to 56, wherein R₃ is heteroaryl.
62. The compound of embodiment 39, wherein the compound is selected from the group consisting of:

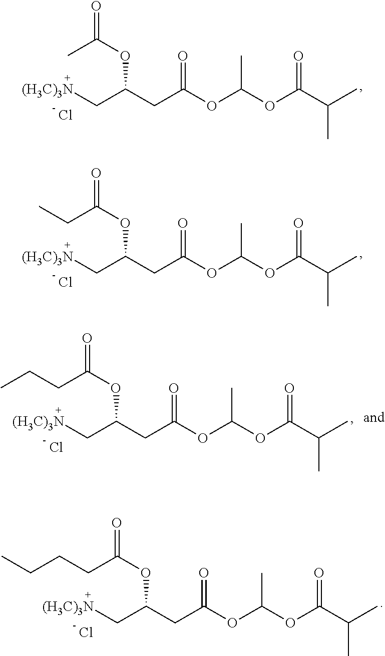

63. A compound of Formula (IV):

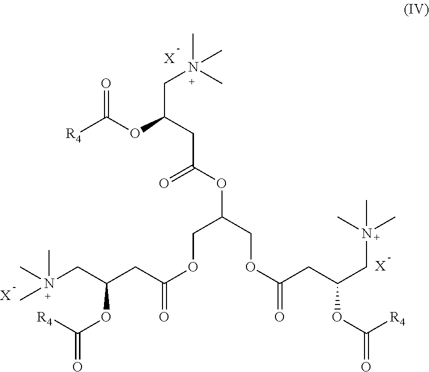

wherein, each R₄ is independently C₁₋₆ alkyl, and

X⁻ is a pharmaceutically acceptable anion.
64. A compound of embodiment 63, wherein R₄ is methyl.
65. A compound of embodiment 63, wherein R₄ is ethyl.
66. A compound of embodiment 63, wherein R₄ is butyl.
67. A pharmaceutical composition comprising any one of compounds 1 to 66 and a pharmaceutically acceptable excipient.
68. A method of treating a mental health disease or disorder, the method comprising administering to a subject in need thereof a therapeutically effective amount of any one of the compounds of embodiments 1 to 66 or a pharmaceutically acceptable salt thereof
69. A method of treating a mental health disease or disorder, the method comprising administering to a subject in need thereof a therapeutically effective amount of the composition of embodiment 68.

1. A compound of Formula (I): 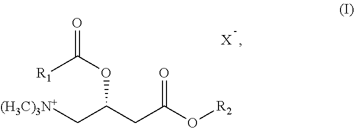
wherein:
R₁ is alkyl, alkenyl, alkynyl, or aryl;
R₂ is alkyl, alkenyl, alkynyl, or aryl; and
X⁻ is a pharmaceutically acceptable anion.

2. The compound of claim 1, wherein R₁ is C₁-C₄ alkyl.

3. The compound of claim 1, wherein R₁ is C₅-C₁₁ alkyl.

4. The compound of claim 1, wherein R₁ is CH(CH₂CH₂CH₃)₂.

5. The compound of claim 1, wherein R₁ is C₁₂-C₂₀ alkyl.

6. The compound of claim 1, wherein R₁ is a long chain fatty acid alkyl including ≥21 carbons, or very long chain fatty acid alkenyl including ≥21 carbons.

7. The compound of claim 6, wherein R₁ is C₂₂-C₃₉ alkyl.

8. The compound of claim 1, wherein R₂ is methyl.

9. The compound of claim 1, wherein R₂ is ethyl.

10. The compound of claim 1, wherein R₂ is n-propyl.

11. The compound of claim 1, wherein R₂ is isopropyl.

12. The compound of claim 1, wherein the compound is selected from the group consisting of:

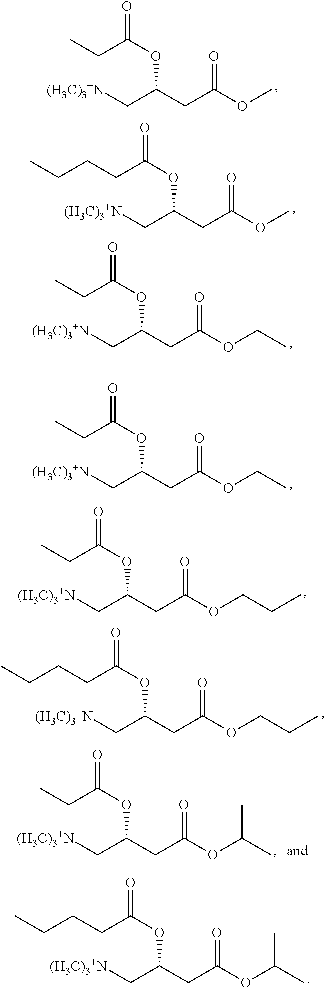

13. A compound of Formula (II): 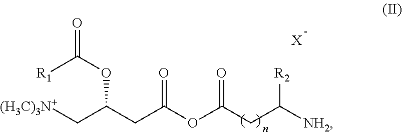
wherein:
R₁ is alkyl, alkenyl, alkynyl or aryl;
R₂ is H, COOH, COORₐ, or CONR_{b}R_{c};
Rₐ is alkyl, cycloalkyl, aryl, or heteroaryl;
R_{b} and R_{c} are independently hydrogen, alkyl, cycloalkyl, aryl, or heteroaryl;
n is an integer from 2-5; and
X⁻ is a pharmaceutically acceptable anion.

14. The compound of claim 13, wherein R₁ is C₁-C₄ alkyl.

15. The compound of claim 13, wherein R₁ is C₅-C₁₁ alkyl.

16. The compound of claim 13, wherein R₁ is CH(CH₂CH₂CH₃)₂.

17. The compound of claim 13, wherein R₁ is C₁₂-C₂₀ alkyl.

18. The compound of claim 13, wherein R₁ is a long chain fatty acid alkyl including ≥21 carbons.

19. The compound of claim 18, wherein R₁ is C₂₂-C₃₆ alkyl.

20. The compound of claim 13, wherein R₂ is hydrogen.

21. The compound of claim 13, wherein R₂ is COOH.

22. The compound of claim 13, wherein n is 2.

23. The compound of claim 13, wherein n is 3

24. The compound of claim 13, wherein n is 4.

25. The compound of claim 13, wherein n is 5.

26. The compound of claim 13, wherein the compound is selected from the the group consisting of:

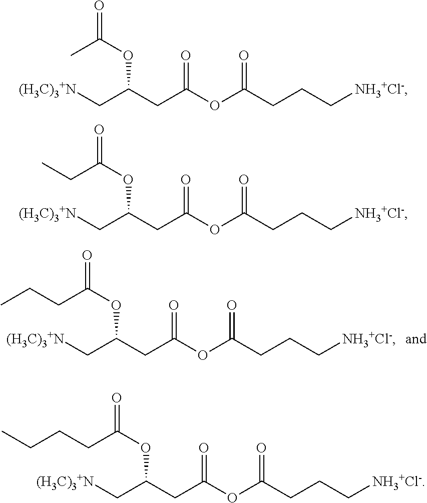

27. A compound of Formula (III): 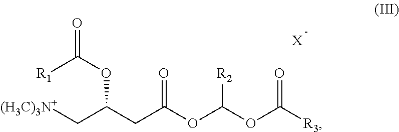
wherein:
R₁ is alkyl, alkynyl, or aryl;
R₂ is alkyl, cycloalkyl, aryl, or heteroaryl;
R₃ is alkyl, cycloalkyl, aryl, or heteroaryl; and
X⁻ is a pharmaceutically acceptable anion.

28. The compound of claim 27, wherein R₁ is C₁-C₄ alkyl.

29. The compound of claim 27, wherein R₁ is C₅-C₁₁ alkyl.

30. The compound of claim 27, wherein R₁ is CH(CH₂CH₂CH₃)₂.

31. The compound of claim 27, wherein R₁ is C₁₂-C₂₀ alkyl.

32. The compound of claim 27, wherein R₁ is a fatty acid alkyl including ≥21 carbons.

33. The compound of claim 27, wherein R₁ is C₂₂-C₃₆ alkyl.

34. The compound of claim 27, wherein R₂ is alkyl.

35. The compound of claim 34, wherein R₂ is methyl.

36. The compound of claim 34, wherein R₂ is isopropyl.

37. The compound of claim 34, wherein R₂ is tert-butyl.

38. The compound of claim 27, wherein R₂ is cycloalkyl.

39. The compound of claim 38, wherein R₂ is cyclopentyl.

40. The compound of claim 27, wherein R₂ is aryl.

41. The compound of claim 40, wherein R₂ is phenyl.

42. The compound of claim 27, wherein R₃ is alkyl.

43. The compound of claim 27, wherein R₃ is aryl.

44. A compound of Formula (IV): 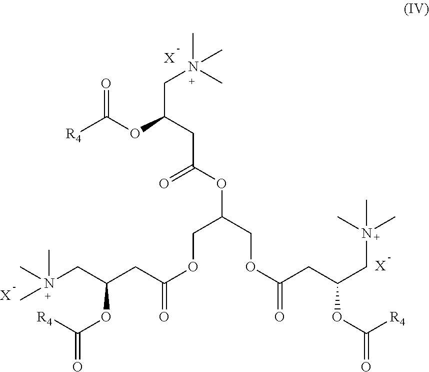
wherein, each R₄ is independently C₁₋₆ alkyl, alkynyl, or aryl, and
X⁻ is a pharmaceutically acceptable anion.

45. A compound of claim 44, wherein R₄ is methyl.

46. A pharmaceutical composition comprising the compound of claim 1 and a pharmaceutically acceptable excipient.

47. A method of treating a mental health disease or disorder in a subject in need thereof, the method comprising administering a therapeutically effective amount of a compound of claim 1 to the subject.
